(19) United States
(12) Patent Application Publication
(10) Pub. No.: US 2002/0155480 A1
(43) Pub. Date: Oct. 24, 2002

(54) Brain tumor diagnosis and outcome prediction

(76) Inventors: Todd R. Golub, Newton, MA (US); Eric S. Lander, Cambridge, MA (US); Scott Pomeroy, Newton, MA (US); Pablo Tamayo, Cambridge, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/066,305

(22) Filed: Jan. 31, 2002

Related U.S. Application Data
(60) Provisional application No. 60/265,482, filed on Jan. 31, 2001.

(51) Int. Cl. C12Q 1/68

(52) U.S. Cl. 435/6

(57) ABSTRACT

Methods for predicting phenotypic classes of brain tumors, such as brain tumor type or treatment outcome, for brain tumor samples based on gene expression profiles are described.

### RELATED APPLICATION

This application claims the benefit of U.S. Provisional Application No. 60/265,482, filed on Jan. 31, 2001.

The entire teachings of the above application are incorporated herein by reference.

### GOVERNMENT SUPPORT

The invention was supported, in whole or in part, by a grant R01NS35701 from the National Institutes of Health. The Government has certain rights in the invention.

### BACKGROUND OF THE INVENTION

Classification of biological samples from individuals is not an exact science. In many instances, accurate diagnoses and safe and effective treatment of a disorder depend on being able to discern biological distinctions among morphologically similar samples, such as tumor samples. The classification of a sample from an individual into particular disease classes has often proven to be difficult, incorrect or equivocal. Typically, using traditional methods such as histochemical analyses, immunophenotyping and cytogenetic analyses, only one or two characteristics of the sample are analyzed to determine the sample's classification, resulting in inconsistent and sometimes inaccurate results. Such results can lead to incorrect diagnoses and potentially ineffective or harmful treatment. Furthermore, important biological distinctions are likely to exist that have yet to be identified due to the lack of systematic and unbiased approaches for identifying or recognizing such classes. Thus, a need exists for an accurate and efficient method for identifying biological classes and classifying samples.

### SUMMARY OF THE INVENTION

Embryonal tumors of the central nervous system (CNS) represent a heterogeneous group of tumors about which little is known biologically, and whose diagnosis, based on morphologic appearance alone, is controversial. Using the methods described herein, brain tumors can be classified using molecular distinctions that discriminate between, for example, medulloblastomas and other brain tumors. Molecular distinctions can also be made, for example, for including primitive neuroectodermal tumors (hereinafter, “PNET”), atypical teratoid/rhabdoid tumors (AT/RT) and malignant gliomas. Further, the clinical outcome of patients (e.g., children) with medulloblastomas is highly predictable based on the gene expression profiles of their tumors at diagnosis.

The present invention relates to one or more sets of informative genes whose expression correlates with a class distinction among brain tumor samples. In a particular embodiment, the class distinction is a brain tumor class distinction, such as a classic medulloblastoma, desmoplastic medulloblastoma, rhabdoid tumor, supratentorial PNET, pineoblastoma or glioblastoma. In another embodiment the class distinction is a treatment outcome or survival class distinction. In yet another embodiment, the class distinction is the effectiveness of drugs or agents for treating, for example, brain tumors.

In one embodiment, the present invention is directed to a method of classifying a brain tumor including the steps of: obtaining a sample of cells derived from a brain tumor; isolating a gene expression product from at least one informative gene from one or more cells in the sample; and determining a gene expression profile of at least one informative gene, wherein the gene expression profile is correlated with a specific brain tumor sub-type. In a particular embodiment, the brain tumor is selected from the group consisting of: medulloblastoma, rhabdoid tumor, primitive neuroectodermal tumor, pineoblastoma or glioblastoma. In one embodiment, the brain tumor type is a medulloblastoma or a glioblastoma. In another embodiment, the medulloblastoma sub-type is classic medulloblastoma or desmoplastic medulloblastoma. In one embodiment, the expression profile comprises expression of Zic or NSCL-1. In one embodiment, the expression profile includes expression of TrkC. In one embodiment, the gene expression product is mRNA. In another embodiment, the gene expression profile is determined utilizing specific hybridization probes. In a specific embodiment, the gene expression profile is determined utilizing oligonucleotide microarrays. In another embodiment, the gene expression product is a polypeptide. In a particular embodiment, the gene expression profile is determined utilizing antibodies. In a particular embodiment, the informative gene can be one or more genes listed in FIGS. 2A-2B, 3A-3B, 5A-5B and 6B-6C. The informative gene can be one or more genes listed in FIGS. 1A-1B.

In another embodiment, the present invention is directed to a method of predicting the efficacy of treating a brain tumor comprising the steps of: obtaining a sample of cells derived from a brain tumor; isolating a gene expression product from at least one informative gene from one or more cells in said sample; and determining a gene expression profile of at least one informative gene, wherein the gene expression profile is correlated with a treatment outcome, thereby classifying the sample with respect to treatment outcome. In a particular embodiment, the brain tumor is selected from the group consisting of: medulloblastoma, rhabdoid tumor, primitive neuroectodermal tumor, pineoblastoma and glioblastoma.. In a particular embodiment the brain tumor type is a medulloblastoma or a glioblastoma. In another embodiment, the medulloblastoma sub-type is classic medulloblastoma or desmoplastic medulloblastoma. The gene expression product can be, for example, mRNA. In one embodiment, the gene expression profile can be determined utilizing specific hybridization probes. The gene expression profile can be determined utilizing oligonucleotide microarrays. In another embodiment, the gene expression product can be a polypeptide. The gene expression profile can thus be determined utilizing antibodies. In a particular embodiment, the predicted treatment outcome can be, for example, survival after treatment. The informative gene can be one or more genes listed in FIGS. 1A-1B. Additionally, the informative gene can be one or more genes listed in FIGS. 2A-2B, 3A-3B, 5A-5B and 6B-6C.

In another embodiment, the present invention is directed to a method of assigning a brain tumor sample to a treatment outcome class, comprising the steps of: determining a weighted vote for one of the classes of one or more informative genes in the sample in accordance with a model built with a weighted voting scheme, such that the magnitude of each vote depends on the expression level of the gene in said sample and on the degree of correlation of the gene's expression with class distinction; and summing the votes to determine the winning class, such that the winning class is the treatment outcome class to which the brain tumor sample is assigned. In a particular embodiment, the weighted voting scheme is:

V_{g}=a_{g}(x_{g}−b_{g}),

wherein V_{g} is the weighted vote of the gene, g; a_{g} is the correlation between gene expression values and class distinction; b_{g}=(μ₁(g)+μ₂(g))/2 is the average of the mean log₁₀ expression value in a first class and a second class; x_{g} is the log₁₀ gene expression value in the sample to be tested; and wherein a positive V value indicates a vote for the first class, and a negative V value indicates a vote for the second class. The informative genes can be any of those listed in FIGS. 1A-1B, FIGS. 2A-2B, FIGS. 3A-3B, FIGS. 5A-5B and FIGS. 6B-6C.

In another embodiment, the present invention is an oligonucleotide microarray having immobilized thereon a plurality of oligonucleotide probes specific for one or more informative genes listed in FIGS. 1A-1B, 2A-2B, 3A-3B, 5A-5B and 6B-6C.

In another embodiment, the present invention is directed to a method for evaluating candidate therapeutic agents (e.g., drugs) for their effectiveness in treating brain tumors comprising: obtaining a sample of cells derived from a brain tumor; isolating a gene expression product from at least one informative gene from one or more cells in said sample; and determining a gene expression profile of at least one informative gene, such that the gene expression profile is correlated with the effectiveness of the drug candidate in treating brain tumors.

In another embodiment, the present invention is directed to a method for monitoring the efficacy of a brain tumor treatment comprising: obtaining samples of cells at various time points derived from a patient being treated; determining the expression profile of the samples; classifying the samples for treatment outcome based on the expression profile; and comparing the treatment outcome class of the samples at various times during treatment, such that the efficacy of brain tumor treatment is determined.

In another embodiment, the present invention is directed to a method for predicting tumorigenesis comprising: obtaining samples of cells at various time points derived from a patient; determining the expression profile of the samples; classifying the samples as tumorigenic or non-tumorigenic based on the expression profile; and comparing the tumorigenic class of the samples at various times, such that the onset of tumorigenesis can be predicted.

### BRIEF DESCRIPTION OF THE FIGURES

The patent or application file contains at least one drawing executed in color. Copies of this patent or patent application publication with color drawings will be provided by the Office upon request and payment of the necessary fee.

FIGS. 1A-1B show a list of medulloblastoma treatment outcome gene markers whose expression is increased (upregulated) in high risk and decreased (downregulated) in low risk individuals, or whose expression is upregulated in low risk and downregulated in high risk individuals. The genes are identified by GenBank Accession number followed by common name.

FIGS. 2A-2B show a list of informative genes whose expression is high in medulloblastoma and low in glioblastoma. The genes are identified by GenBank Accession number followed by common name.

FIGS. 3A-3B show a list of informative genes whose expression is low in medulloblastoma and high in glioblastoma. The genes are identified by GenBank Accession number followed by common name.

FIGS. 4A-4E are depictions of methods and data obtained in classifying embryonal brain tumors by gene expression. FIG. 4A shows representative photomicrographs of embryonal and non-embryonal tumors: a) classic medulloblastoma, b) desmoplastic medulloblastoma, c) supratentorial primitive neuroectodermal tumor (PNET), d) atypical teratoid/rhabdoid tumor (AT/RT; arrow indicates rhabdoid cell morphology), and e) glioblastoma with pseudopalisading necrosis (n). FIG. 4B is a schematic representation of principal component analysis (PCA) of tumor samples using all genes exhibiting variation across the dataset. The axes represent the 3 linear combinations of genes that account for the majority of the variance in the original dataset (see Supplementary Information Section I and III; http://www.genome.wi.mit.edu/MPR/CNS). FIG. 4C is a schematic representation of PCA using 50 genes selected by signal-to-noise metric to be most highly associated each tumor type (the top 10 for each tumor are listed in FIG. 4E). FIG. 4D is a schematic representation of clustering of tumor samples by hierarchical clustering using all genes exhibiting variation across the dataset. FIG. 4E is a graphical representation of signal-to-noise rankings of genes comparing each tumor type to all other types combined (see Supplementary Information Section I; http://www.genome.wi.mit.edu/MPR/CNS). For each gene, red indicates high level of expression relative to the mean, blue indicates low level of expression relative to the mean.

FIGS. 5A and 5B are graphical representations of differential expression of genes in classic versus desmoplastic medulloblastomas. Depict are data used to rank Genes by the signal-to-noise metric according to their correlation with the classic vs. desmoplastic distinction. Genes shown are those more highly correlated with the distinction than 99% of permutations of the class labels (p<0.01; see Supplementary Information Section III; http://www.genome.wi.mit.edu/MPR/CNS; the entire teachings of which are incorporated herein by reference). GenBank accession numbers and gene descriptions are shown. Genes regulated by Shh are shown at right.

FIG. 6A-6C are graphical representations of data used in predicting medulloblastoma outcome by gene expression profiling. FIG. 6A is a graphical representation of Kaplan-Meier overall survival curves for patients predicted to survive and patients predicted to be treatment failures using an 8-gene k-NN model (P=0.000003, log rank test). FIGS. 6B and 6C are graphical and tabular representations of fifty genes most highly associated with favorable outcome (FIG. 6B) or with treatment failure (FIG. 6C) according to the signal-to-noise metric. Samples are further sorted according to their membership in the two unsupervised SOM-derived clusters (C0, C1). Class C1 tumors are notable for their high ribosomal content. The 8 genes most frequently used by the k-NN outcome predictor are indicated in bold.

### DETAILED DESCRIPTION OF THE INVENTION

Classification of biological samples from individuals is not an exact science. In many instances, accurate diagnosis and safe and effective treatment of a disorder depend on being able to discern biological distinctions among morphologically similar samples, such as tumor samples. The classification of a sample from an individual into particular disease classes has often proven to be difficult, incorrect or equivocal. Typically, using traditional methods such as histochemical analyses, immunophenotyping and cytogenetic analyses, only one or two characteristics of the sample are analyzed to determine the sample's classification. As differences between classes of sample types might amount to differences in the expression of a handful of genes out of the thousands that are expressed in cells, monitoring only one or two genes results in inconsistent and sometimes inaccurate results. This limitation is augmented by the fact that important biological distinctions are likely to exist that have yet to be identified. Inaccurate results can lead to incorrect diagnoses and potentially ineffective or harmful treatment. Thus, a need exists for an accurate and efficient method for identifying biological classes and classifying samples. The present invention is directed to methods for predicting phenotypic classes of brain tumors, such as brain tumor type or treatment outcome, for brain tumor samples based on gene expression profiles are described.

Embryonal tumors of the central nervous system (CNS) represent a heterogeneous group of tumors about which little is known biologically, and whose diagnosis, based on morphologic appearance alone, is controversial. Medulloblastomas, for example, are the most common malignant brain tumor of childhood, but their pathogenesis is unknown, their relationship to other embryonal CNS tumors is debated (Rorke, L., 1983. J. Neuropathol. Exp. Neurol., 42:1-15; Kadin, M. et al., 1970. J. Neuropath. Exp. Neurol., 29:583-600), and patients' response to therapy is difficult to predict (Packer, R. et al., 1999. J. Clin. Oncol., 17:2127-2136). These problems were addressed by developing a classification system based on DNA microarray gene expression data derived from 99 patient samples. Medulloblastomas are demonstrably molecularly distinct from other brain tumors including primitive neuroectodermal tumors (PNET), atypical teratoid/rhabdoid tumors (AT/RT) and malignant gliomas. Previously unrecognized evidence supporting the derivation of medulloblastomas from cerebellar granule cells through activation of the Sonic Hedgehog (Shh) pathway was also revealed. Further, the clinical outcome of children with medulloblastomas is highly predictable based on the gene expression profiles of their tumors at diagnosis.

The present invention relates to methods for classifying a sample according to the gene “expression profile” of the sample. As used herein, an “expression profile” refers to the level or amount of gene expression of one or more genes (e.g., informative genes) in a given sample of cells at one or more time points. In one embodiment, the present invention is directed to a method of classifying a brain tumor sample with respect to a phenotypic effect, e.g., brain tumor type or predicted treatment outcome, including the steps of isolating a gene expression product from one or more cells in the sample and determining a gene expression profile for at least one informative gene, wherein the gene expression profile is correlated with a phenotypic effect, thereby classifying the sample with respect to phenotypic effect. This embodiment is directed to the assessment of “informative genes,” used herein to refer to a gene or genes whose expression correlates with a particular phenotype. Expression profiles obtained for informative genes can be used to determine particular sample cell phenotypes. Samples can be classified according to their broad expression profile, or according to the expression levels of particular informative genes.

According to methods of the invention, samples can be classified as belonging to (or derived from) a particular type of brain tumor. For example, a sample can be classified as derived from a classic medulloblastoma, desmoplastic medulloblastoma, rhabdoid tumor, supratentorial primitive neuroectodermal tumor (hereinafter, “PNET”), pineoblastoma or glioblastoma. Class distinctions among these brain tumor sub-types are not readily apparent using traditional analytic methods for sample classification.

In addition to brain tumor sub-type classifications, samples can be classified according to their susceptibility to particular treatments. For example, cell samples derived from brain tumors can be classified according to their response to particular treatments where the response can be reduction of tumor size, repression of cell growth, or survival rate of the patient from whom the sample was derived. In a preferred embodiment the treatment outcome is survival. That is, a sample can be classified as belonging to a high risk class (e.g., a class with poor prognosis for survival after treatment) or a low risk class (e.g., a class with good prognosis for survival after treatment). Duration of illness, severity of symptoms and eradication of disease can also be used as the basis for classifying samples.

As used herein, “gene expression products” are proteins, polypeptides, or nucleic acid molecules (e.g., mRNA, tRNA, rRNA, or cRNA) that result from transcription or translation of genes. The present invention can be effectively used to analyze proteins, peptides or nucleic acid molecules that are the result of transcription or translation. The nucleic acid molecule levels measured can be derived directly from the gene or, alternatively, from a corresponding regulatory gene or regulatory sequence element. All forms of gene expression products can be measured. Additionally, variants of genes and gene expression products including, for example, spliced variants and polymorphic alleles, can be measured. Similarly, gene expression can be measured by assessing the level of protein or derivative thereof translated from mRNA. The sample to be assessed can be any sample that contains a gene expression product. Suitable sources of gene expression products, e.g., samples, can include intact cells, lysed cells, cellular material for determining gene expression, or material containing gene expression products. Examples of such samples are brain, blood, plasma, lymph, urine, tissue, mucus, sputum, saliva or other cell samples. Methods of obtaining such samples are known in the art. In a preferred embodiment, the sample is derived from an individual who has been clinically diagnosed as having a brain tumor.

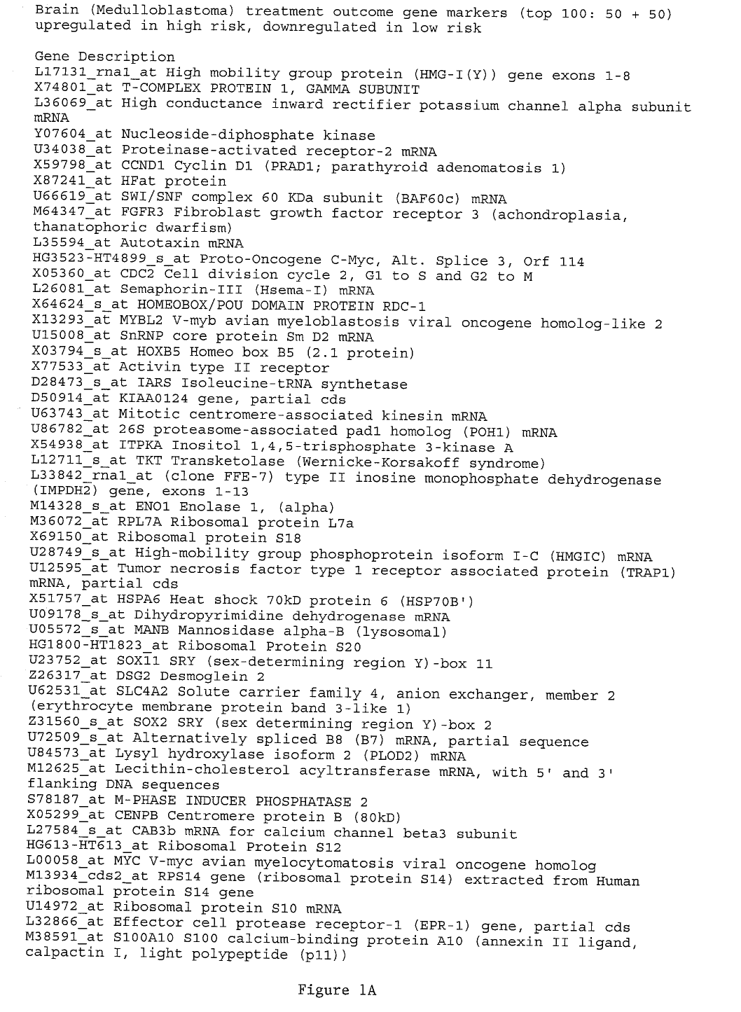

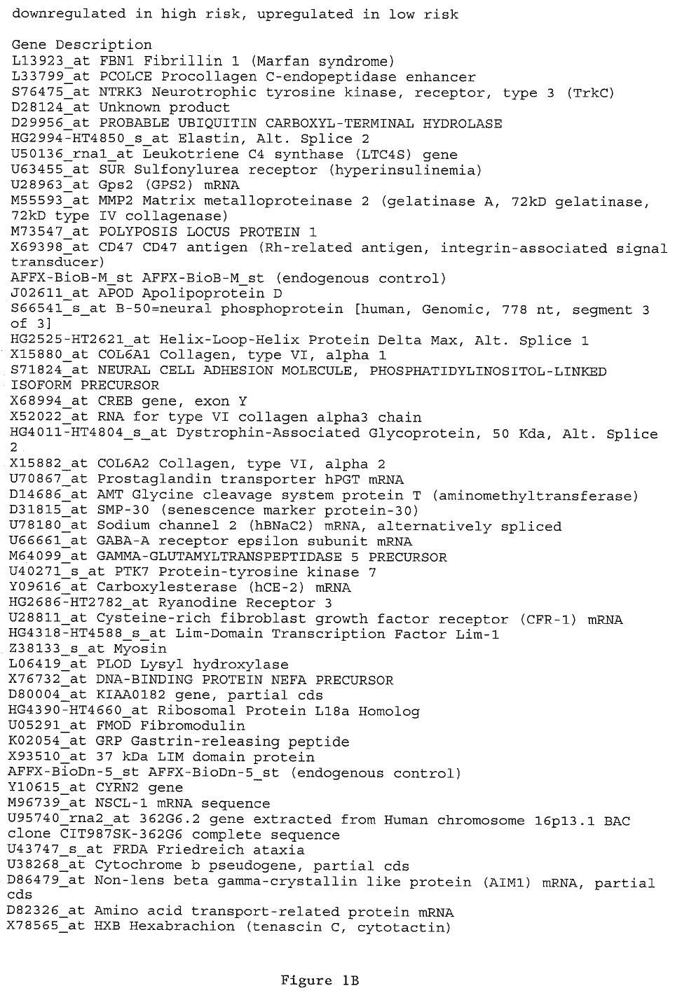

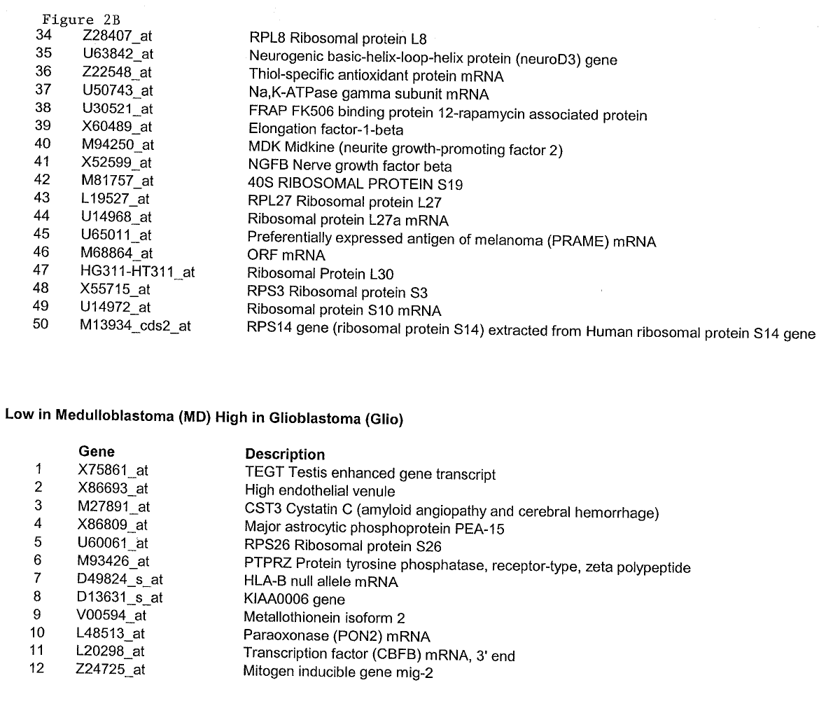

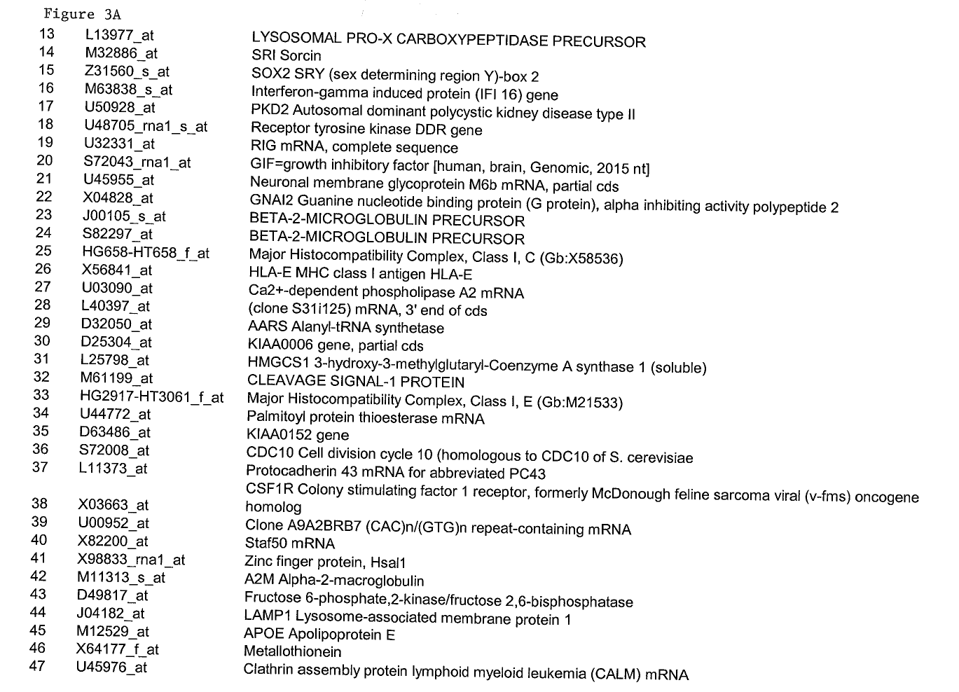

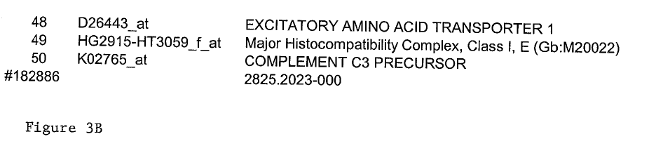

Genes that are particularly relevant for classification, i.e., demonstrate a different expression profile in different classification categories, have been identified as a result of work described herein and are shown in FIGS. 1A-1B, 2A-2B, 3A-3B, 5A-5B and 6B-6C. The genes that are relevant for classification are referred to herein as “informative genes.” Not all informative genes for a particular class distinction must be assessed in order to classify a sample. Similarly, the set of informative genes that characterize one phenotypic effect may or may not be the same as the set of informative genes for a different phenotypic effect. For example, a subset of the informative genes that demonstrate a high correlation with a class distinction can be used in classifying brain tumor sub-types. This subset can be, for example, one or more genes, 5 or more genes, 10 or more genes, 25 or more genes, or 50 or more genes. The informative genes that characterize other classification categories such as, for example, treatment outcome, can be the same or different from the informative genes that characterize brain tumor sub-types. Typically the accuracy of the classification increases with the number of informative genes that are assessed.

In one embodiment, the gene expression product is a protein or polypeptide. In this embodiment the determination of the gene expression profile is made using techniques for protein detection and quantitation known in the art. For example, antibodies that specifically interact with the protein or polypeptide expression product of one or more informative genes can be obtained using methods that are routine in the art. The specific binding of such antibodies to protein or polypeptide gene expression products can be detected and measured by methods known in the art.

A gene expression profile can comprise data for one or more genes and can be measured at a single time point or over a period of time. Phenotype classification (e.g., treatment outcome, brain tumor type) can be made by comparing the gene expression profile of the sample to one or more gene expression profiles (e.g., in a database). Specific classifications involve comparing common informative genes whose expression is included in both expression profiles. Informative genes include, but are not limited to, those shown in FIGS. 1A-1B, 2A-2B, 3A-3B, 5A-5B and 6B-6C. Using the methods described herein, expression of numerous genes can be measured simultaneously, thus avoiding problems encountered with traditional classification methods that monitor only a few aspects of classification categories.

In a preferred embodiment, the gene expression product is mRNA and the gene expression levels are obtained, e.g., by contacting the sample with a suitable microarray on which probes specific for all or a subset of the informative genes have been immobilized, and determining the extent of hybridization of the nucleic acid in the sample to the probes on the microarray. Such microarrays are also within the scope of the invention. Examples of methods of making oligonucleotide microarrays are described, for example, in WO 95/11995. Other methods are readily known to the skilled artisan.

Once the gene expression levels of the sample are obtained, the levels are compared or evaluated against a model or control sample(s), and then the sample is classified. The evaluation of the sample determines whether or not the sample is assigned to a particular phenotypic class.

The gene expression value measured or assessed is the numeric value obtained from an apparatus that can measure gene expression levels. Gene expression levels refer to the amount of expression of the gene expression product, as described herein. The values are raw values from the apparatus, or values that are optionally re-scaled, filtered and/or normalized. Such data is obtained, for example, from a GeneChip® probe array or Microarray (Affymetrix, Inc.; U.S. Pat. Nos. 5,631,734, 5,874,219, 5,861,242, 5,858,659, 5,856,174, 5,843,655, 5,837,832, 5,834,758, 5,770,722, 5,770,456, 5,733,729, 5,556,752, all of which are incorporated herein by reference in their entirety), and the expression levels are calculated with software (e.g., Affymetrix GENECHIP software). Nucleic acids (e.g., mRNA) from a sample that has been subjected to particular stringency conditions hybridize to the probes on the chip. The nucleic acid to be analyzed (e.g., the target) is isolated, amplified and labeled with a detectable label, (e.g., ³²P or fluorescent label) prior to hybridization to the arrays. After hybridization, the arrays are inserted into a scanner that can detect patterns of hybridization. These patterns are detected by detecting the labeled target now attached to the microarray, e.g., if the target is fluorescently labeled, the hybridization data are collected as light emitted from the labeled groups. Since labeled targets hybridize, under appropriate stringency conditions known to one of skill in the art, specifically to complementary oligonucleotides contained in the microarray, and since the sequence and position of each oligonucleotide in the array are known, the identity of the target nucleic acid applied to the probe is determined.

Quantitation of gene profiles from the hybridization of a labeled mRNA/DNA microarray can be performed by scanning the microarray to measure the amount of hybridization at each position on the microarray with an Affymetrix scanner (Affymetrix, Santa Clara, Calif.). For each stimulus a time series of mRNA levels (C={C1,C2,C3, . . . Cn}) and a corresponding time series of mRNA levels (M={M1,M2,M3, . . . Mn}) in control medium in the same experiment as the stimulus is obtained. Quantitative data is then analyzed. “Cᵢ” and “Mᵢ” are defined as relative steady-state mRNA levels, where “i” refers to the i^{th} time point and n to the total number of time points of the entire timecourse. “μM” and “σM” are defined as the mean and standard deviation of the control time course, respectively. Hybridization analysis using microarray is only one method for obtaining gene expression values. Other methods for obtaining gene expression values known in the art or developed in the future can be used with the present invention. Once the gene expression values are determined, the sample can be classified.

The correlation between gene expression and class distinction can be determined using a variety of methods. Methods for defining classes and classifying samples are described, for example, in U.S. patent application Ser. No. 09/544,627, filed Apr. 6, 2000 by Golub et al., the teachings of which are incorporated herein by reference in their entirety. The information provided by the present invention, alone or in conjunction with other test results, aids in sample classification.

In one embodiment, the sample is classified using a weighted voting scheme. The weighted voting scheme advantageously allows for the classification of a sample on the basis of multiple gene expression values. In a preferred embodiment the sample is a brain tumor sample derived from a patient, e.g., a medulloblastoma or glioblastoma patient sample. In a preferred embodiment the sample is classified as belonging to a particular treatment outcome class. In another embodiment the gene is selected from a group of informative genes, including, but not limited to, the genes listed in FIGS. 1A-1B, FIGS. 2A-2B, 3A-3B, 5A-5B and 6B-6C.

One aspect of the present invention is a method for assigning a sample to a known or putative class, e.g., a brain tumor treatment outcome class, comprising determining a weighted vote of one or more informative genes (e.g., greater than 5, 10, 20, 30, 40 or 50 genes) for one of the classes in accordance with a model built with a weighted voting scheme, wherein the magnitude of each vote depends on the expression level of the gene in the sample and on the degree of correlation of the gene's expression with class distinction; and summing the votes to determine the winning class. The weighted voting scheme is:

V_{g}=a_{g}(x_{g}−b_{g}),

wherein “V_{g}” is the weighted vote of the gene, g; “a_{g}” is the correlation between gene expression values and class distinction, P(g,c), as defined herein; “b_{g}=(μ₁(g) ₂(g))/2” is the average of the mean log₁₀ expression value in a first class and a second class; “x_{g}” is the log₁₀ gene expression value in the sample to be tested; and wherein a positive V value indicates a vote for the first class, and a negative V value indicates a negative vote for the class.

A prediction strength can also be determined, wherein the sample is assigned to the winning class if the prediction strength is greater than a particular threshold, e.g., 0.3. The prediction strength is determined by:

(V_{win}−Vₗₒₛₑ)/(V_{win}+Vₗₒₛₑ),

wherein “V_{win}” and “Vₗₒₛₑ” are the vote totals for the winning and losing classes, respectively.

As a consequence of the identification of informative genes for the prediction of treatment outcome, the present invention provides methods for determining a treatment plan for an individual. That is, a determination of the brain tumor class or treatment outcome class to which the sample belongs may dictate that a treatment regimen be implemented. For example, once a health care provider knows which treatment outcome class the sample, and therefore, the individual from which it was obtained, belongs, the health care provider can determine an adequate treatment plan for the individual. For example, in the treatment of a patient whose gene expression profile as determined by the present invention correlates with a poor prognosis, a health care provider could utilize a more aggressive treatment for the patient, or at minimum provide the patient with a realistic assessment of his or her prognosis.

The present invention also provides methods for monitoring the effect of a treatment regimen in an individual by monitoring the gene expression profile for one or more informative genes. For example, a baseline gene expression profile for the individual can be determined, and repeated gene expression profiles can be determined at time points during treatment. A shift in gene expression profile from a profile correlated with poor treatment outcome to profile correlated with improved treatment outcome is evidence of an effective therapeutic regimen, while a repeated profile correlated with poor treatment outcome is evidence of an ineffective therapeutic regimen.

Alternatively, samples could be obtained from an individual and the gene expression profile of one or more genes can be monitored in order to predict the onset of tumorigenesis. This application of the invention would involve comparing gene expression profiles from the individual at different points in the individual's life and classifying samples as tumorigenic or non-tumorigenic based on the gene expression profile of one or more informative genes. As used herein, “tumorigenic” refers to a state that is generally understood to indicate tumor growth or potential tumor growth.

In addition to monitoring the effectiveness of a particular treatment, the present invention can be applied to screen potential drug candidates for their efficacy in treating brain tumors. In this embodiment, a sample's expression profile is compared before and after treatment with the candidate drug, wherein a shift in the gene expression profile in the treated sample from a profile correlated with poor treatment outcome to a profile correlated with improved treatment outcome is evidence for the efficacy of the drug in treating brain tumors.

The present invention also provides information regarding the genes that are important in brain tumor treatment response, thereby providing additional targets for diagnosis and therapy. It is clear that the present invention can be used to generate databases comprising informative genes that will have many applications in medicine, research and industry; such databases are also within the scope of the invention.

The invention will be further described with reference to the following non-limiting examples. The teachings of all the patents, patent applications and all other publications and websites cited herein are incorporated by reference in their entirety.

### EXEMPLIFICATION

### EXAMPLE 1

Treatment Outcome Prediction

A gene expression-based predictor of medulloblastoma patient response to treatment was built by analyzing patient samples. RNA obtained from patients was analyzed on Affymetrix (Santa Clara, Calif.) oligonucleotide arrays containing probes for 6817 genes as previously described (Tamayo, P. et al., 1999. Proc. Natl. Acad. Sci. USA. 96:2907-2912). In addition to the weighted voting method described, a “k-Nearest Neighbors” (k-NN) algorithm was applied. The k-NN algorithm makes no assumptions about the data and “memorizes” the training set. To predict a new sample it computes the distance of the new sample to each sample in the memorized training set. Thus, each of the k closest samples will have an associated class. The algorithm sets the class of the new data point to the majority class appearing in the k closest training set samples. In our molecular classification problems, a large set of features must be considered, and, therefore, a feature selection process was performed by which the k-NN algorithm is fed only the features with higher correlation with the target class. This feature selection is done by sorting the features according to the same signal-to-noise statistic used in the weighted voting algorithm. Other variations of the algorithm were also used, which include different ways to weight the samples in the training set. Algorithmically the two choices used are- weighting the neighbors according to Euclidean distance, and the rank (k) from the new sample.

As a result of these analyses a set of informative genes was identified as shown in FIGS. 1A-1B. These genes show a significant correlation with treatment outcome (e.g., patient survival). Utilizing these genes patient survival can be predicted with high accuracy (p<0.004), even among patients within a single clinical risk group whose prognosis is otherwise indeterminate.

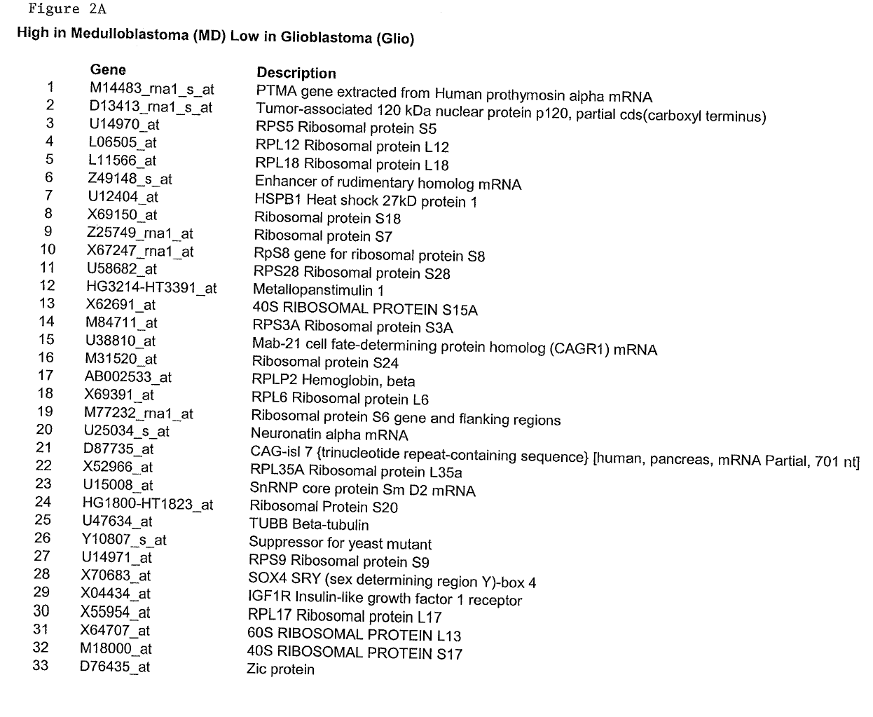

Similar analyses were performed to identify genes that are informative for the medulloblastoma/glioblastoma distinction. As a result of these analyses, a set of informative genes was identified as shown in FIGS. 2A-2B, 3A-3B, 5A-5B and 6B-6C.

### EXAMPLE 2

Prediction of Central Nervous System Embryonal Tumor Outcome Based on Gene Expression.

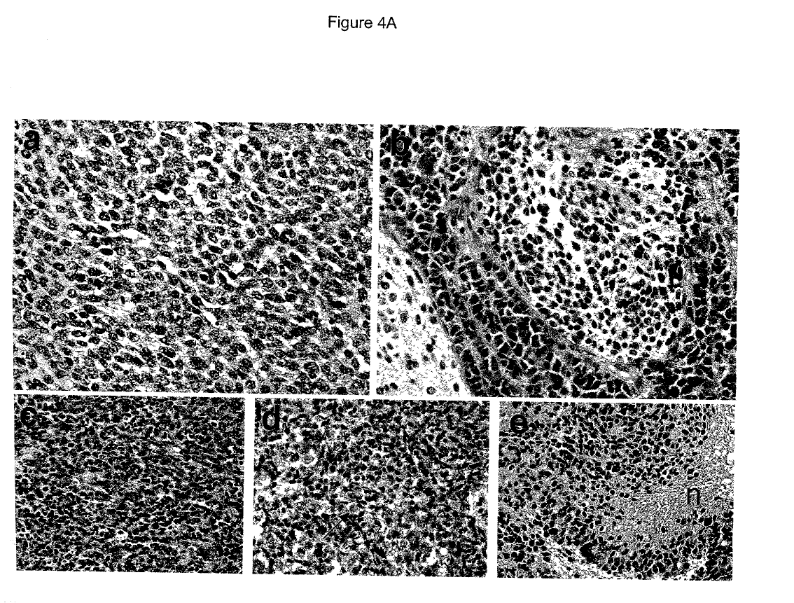

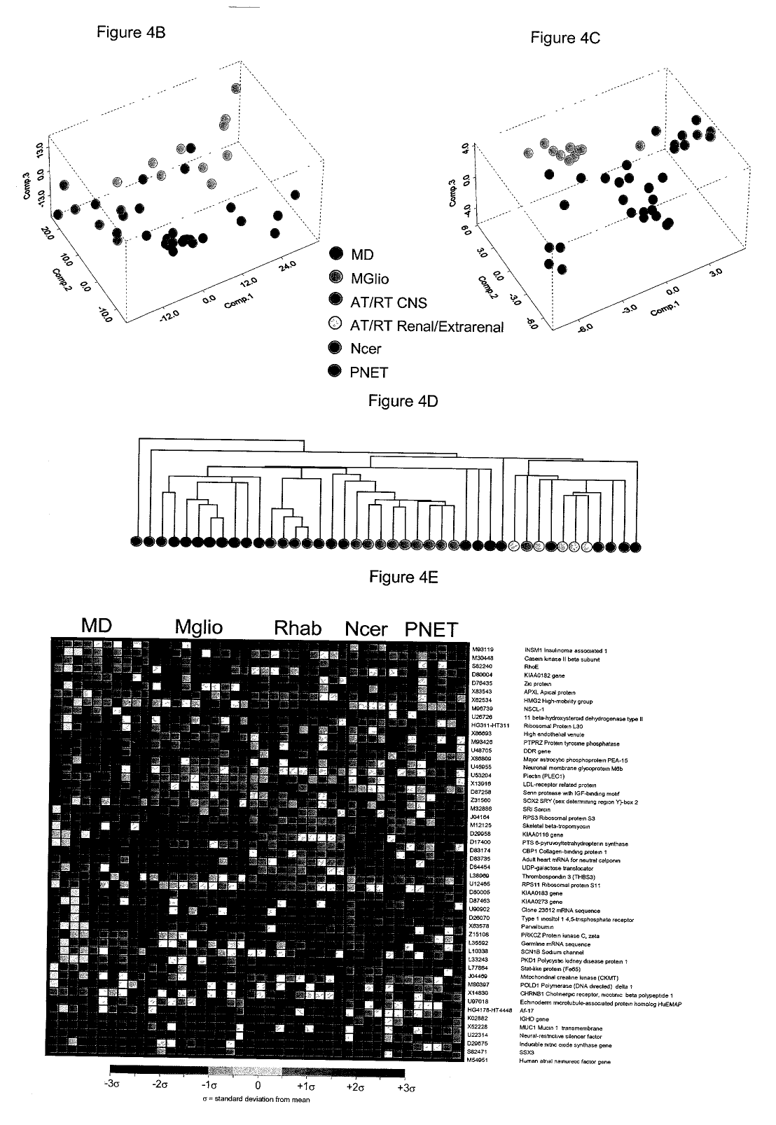

The problem of distinguishing different embryonal CNS tumors from each other was addressed. This is important because the classification of these tumors based on histopathological appearance is debated (FIG. 4A). Some argue that medulloblastomas are part of a larger class of PNETs arising from a common cell type in the subventricular germinal matrix, whereas others believe that they arise from cerebellar granule cell progenitors (Rorke, L., 1983. J. Neuropathol. Exp. Neurol., 42:1-15; Kadin, M. et al., 1970. J. Neuropath. Exp. Neurol., 29:583-600). To begin to generate a molecular taxonomy of CNS embryonal tumors, the gene expression profiles of 42 patient samples were analyzed (Set A: 10 medulloblastomas, 5 CNS AT/RT, 5 renal and extrarenal rhabdoid tumors, and 8 supratentorial PNETs, as well as 10 non-embryonal brain tumors (malignant glioma) and 4 normal human cerebella). RNA extracted from frozen specimens was analyzed with oligonucleotide microarrays containing probes for 6817 genes. The gene expression data are available in “Section II” of “Supplementary Information” (http://www.genome.wi.mit.edu/MPR/CNS).

To determine whether the different types of tumors could be molecularly distinguished, a method of data reduction known as “Principal Component Analysis” in which the high dimensionality of the data was reduced to 3 viewable dimensions representing linear combinations of variables (genes) that account for the majority of the variance in the original dataset was used (FIGS. 4B; Mardia, K. et al., 1979. Multivariate Analysis. Academic Press London.). Normal brain was easily separable from the brain tumors and the different tumor types were similarly separable. Separation of tumor types was also seen using hierarchical clustering (FIG. 4D; Eisen, M. et al., 1998. Proc. Natl. Acad. Sci. USA, 95:14863-14868). A more appropriate strategy for distinguishing known tumor types, however, is to use supervised learning methods to identify the genes most highly correlated with the tumor type distinctions (FIG. 4C and 4E). Analysis of 1,000 random permutations of the data failed to yield a separation of tumor classes to the extent observed in FIG. 4C, indicating that the observed gene expression patterns could not be explained by chance (Supplementary Information Section III; http://www.genome.wi.mit.edu/MPR/CNS). The robustness of these markers for classification was further investigated using a Weighted Voting algorithm and evaluated by cross validation testing (Golub, T. et al., 1999. Science, 286:531-537). Correct classification of the tumors was achieved with accuracy (35 of 42 correct classifications, P<10⁻¹⁰ compared to random classification; Supplementary Information Section III; http://www.genome.wi.mit.edu/MPR/CNS).

As expected, malignant gliomas were clearly separable from medulloblastomas, reflecting the derivation of gliomas from cells of non-neuronal origin. Consistent with this, the gliomas expressed genes typical of the astrocytic and oligodendrocytic lineage (PEA-15, SOX2, PMP-2, Olig-2, TrkB kinase-negative splice variant, S-100, GFAP), genes related to metabolism (fructose 2,6-bisphosphatase, glutamate dehydrogenase), and genes involved in cell differentiation (ID2, GDF-1, TYK2; FIG. 4E and Supplementary Information Section III; http://www.genome.wi.mit.edu/MPR/CNS). Unexpectedly, the medulloblastomas form a cluster that is also separate from the PNETs (FIG. 4C), supporting the notion that these two classes of embryonal tumors are indeed molecularly distinct. Among the genes most highly correlated with the medulloblastoma class were Zic and NSCL-1, encoding transcription factors that have been shown to be specific for cerebellar granule cells (FIG. 4E; Aruga, J. et al., 1994. J. Neurochem., 63:1880-1890; Yokota, N. et al., 1996. Cancer Res., 56:377-383). This result suggests that medulloblastomas, but not PNETs, arise from cerebellar granule cells, or alternatively, have activated the transcriptional program of cerebellar granule cells.

Accurate identification of AT/RT is also important because patients with these tumors have an extremely poor prognosis. AT/RT arise either in the CNS or in other organs such as the kidney, where they are referred to as rhabdoid tumors. Most tumors harbor hSNF5/INI1 mutations, but it is unknown whether AT/RT arising in different anatomical locations are molecularly distinct (Rorke, L. et al., 1996. J. Neurosurg., 85:56-65; Biegel, J. et al., 1999. Cancer Res., 59:74-79; Versteege, I. et al., 1998. Nature, 394:203-6). As shown in FIG. 4C, the AT/RT and rhabdoid tumors were clearly distinguishable from the other tumor types in the study. Strikingly, the CNS AT/RT and abdominal rhabdoid tumors were molecularly similar despite having arisen in different anatomical locations. This finding supports the notion that they arise from a similar cell of origin. Alternatively, a common mechanism of transformation yield similar transcriptional programs in cells of distinct origin. Markers of the AT/RT/rhabdoid distinction include genes specifically expressed during myogenesis, including skeletal β-tropomyosin, neutral calponin, NF-AT3, myosin regulatory light chain (FIG. 4E and Supplementary Information Section III; http://www.genome.wi.mit.edu/MPR/CNS). This finding is consistent with the notion that the tumors have a mesenchymal origin.

Another topic to be addressed concerned the molecular heterogeneity within a single tumor type, e.g., medulloblastoma. The major histological subclass of medulloblastoma is desmoplastic medulloblastoma, although its diagnosis is highly subjective (FIG. 4A). Desmoplastic medulloblastoma is of interest because it is seen with high frequency in patients with Gorlin's syndrome, a rare autosomal dominant disorder resulting from mutation of the Sonic hedgehog (Shh) receptor PTCH (Hahn, H. et al., 1996. Cell, 85:841-851; Johnson, R. et al., 1996. Science, 272:1668-1671). Whether dysregulation of the Shh pathway, known to be mitogenic for cerebellar granule cells, is also involved in the pathogenesis of sporadic desmoplastic medulloblastoma, has been debated (Pietsch, T. et al., 1997. Cancer Res., 57:2085-2088; Raffel, C. et al., 1997. Cancer Res., 57:842-845; Xie, J. et al., 1997. Cancer Res., 57:2369-2372; Wechsler-Reya, R. and Scott, M., 1999. Neuron, 22:103-114; Wetmore, C. et al., 2000. Cancer Res., 60:2239-2246).

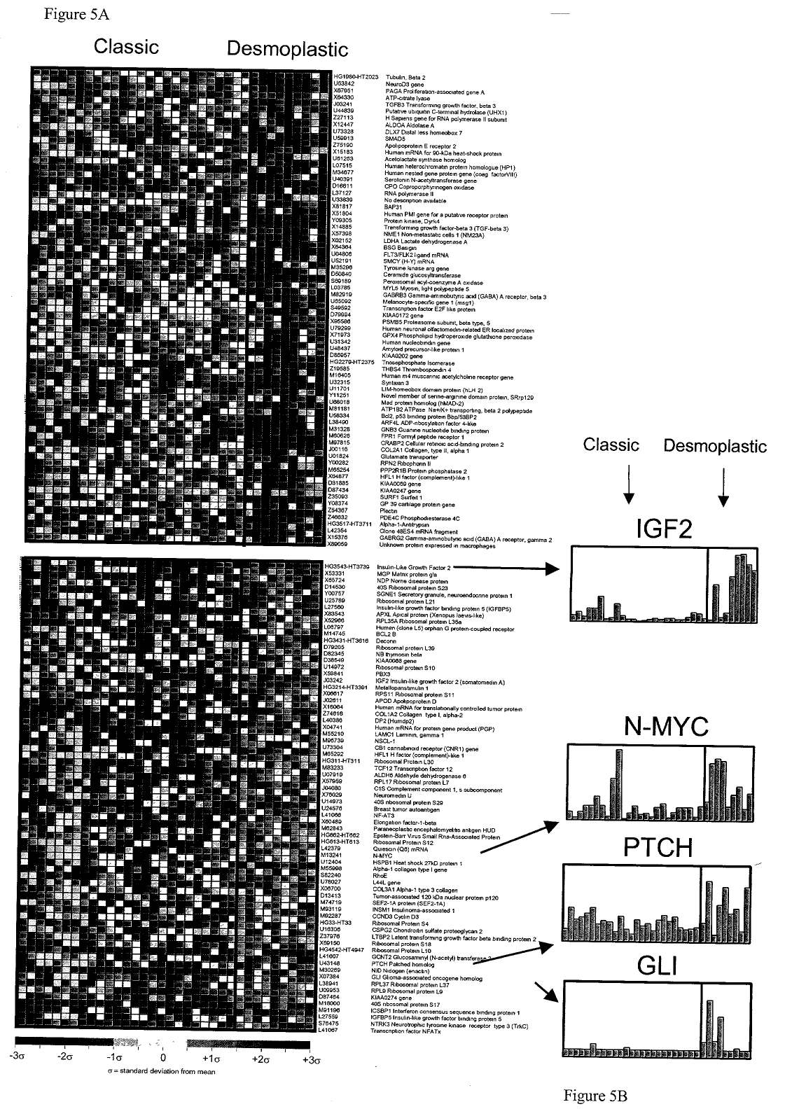

To determine whether desmoplastic and classic medulloblastoma are distinguishable by gene expression, 34 medulloblastoma samples (Set B) whose histology was scored using World Health Organization criteria were analyzed (Giangaspero, F. et al., 2000. Medulloblastoma. In: Kleihues, P. and Cavenee, W. (eds.). World Health Organization Histological Classification of Tumours of the Nervous System. Lyon: International Agency for Research on Cancer, pp. 129-137). As shown in FIGS. 5A and 5B, a sharp and statistically significant gene expression signature of desmoplastic histology was evident, and this signature was sufficient for correct classification of 33 of 34 tumors (P=8.6×10⁻⁷ compared to random classification, Supplementary Information Section III; http://www.genome.wi.mit.edu/MPR/CNS). Strikingly, among the genes most highly correlated with desmoplastic medulloblastoma were PTCH (itself a transcriptional target of Shh) as well as two other Shh downstream targets: Gli and N-Myc (Murone, M. et al., 1999. Curr. Biol., 28:76-84). Furthermore, IGF2 expression was correlated with desmoplastic histology, and its expression is known to be essential for Shh-mediated tumorigenesis in mice (Hahn, H. et al., 2000. J. Biol. Chem., 275:28341-28344). Taken together, the transcriptional profiling indicates that sporadic desmoplastic medulloblastomas, like Gorlin's syndrome-associated tumors, are characterized by activation of Shh signaling pathway, further supporting the suspicion that Shh dysregulation may be important in the pathogenesis of medulloblastoma.

A clinical challenge concerning medulloblastoma is the highly variable response of patients to therapy. Whereas some patients are cured by chemotherapy and radiation, others have progressive disease. Currently, the only prognostic factor used in clinical practice is tumor staging, a reflection of postoperative tumor size and the presence of metastases. Unfortunately, staging-based prognostication is imperfect in that many patients with low stage disease still succumb to their disease. There are currently no molecular markers of outcome used in clinical practice for any brain tumor. High levels of expression of the neurotrophin-3 receptor (TrkC), however, have been reported to correlate with a favorable medulloblastoma outcome, suggesting a molecular basis of medulloblastoma outcome variability (Segal, R. et al., 1994. Proc. Natl. Acad. Sci. USA, 91:12867-12871; Kim, J. et al., 1999. Cancer Res., 59:711-719; Grotzer, M. et al., 2000. J. Clin. Oncol., 18:1027-1035).

To explore the heterogeneity in medulloblastoma treatment response, the analysis was expanded to include 60 similarly treated patients from whom biopsies were obtained prior to receiving treatment, and for whom clinical follow-up was available (Set C). Clustering methods were first used to determine if they would identify biologically distinct subsets of the tumors. The tumors were clustered into two groups using Self-Organizing Maps (SOMs), an unsupervised algorithm that groups samples into a predetermined number of clusters based on their gene expression patterns (Golub, T. et al., 1999. Science, 286:531-537; Tamayo, P. et al., 1999. Proc. Natl. Acad. Sci. USA, 96:2907-2912). The genes most highly correlated with the SOM clusters were primarily ribosomal protein-encoding genes (Supplementary Information Section III; http://www.genome.wi.mit.edu/MPR/CNS), suggesting differences in ribosome biogenesis. Blinded electron microscopic examination of 9 samples by 3 observers confirmed that tumors falling into the cluster characterized by high expression of ribosomal protein genes indeed contained higher numbers of ribosomes (P=0.03, Fisher exact test). The next question was whether the SOM-derived clusters were correlated with patient survival. No statistically significant difference in the proportion of survivors versus treatment failures in each cluster was observed (Fisher Exact Test P=0.1; Supplementary Information Section III; http://www.genome.wi.mit.edu/MPR/CNS). A supervised learning gene expression-based outcome predictor was developed in which the classifier ‘learns’ the distinction between patients who are alive following treatment (‘survivors’) compared to those who succumbed to their disease (‘failures’; minimum follow-up 24 months for surviving patients; overall median 41.5 months).

Additionally, a k-Nearest Neighbors (k-NN) algorithm was used (Dasarathy V. (ed), Nearest Neighbor (NN) Norms: NN Pattern Classification Techniques. IEEE computer society press, Los Alamitos, Calif., December 1991. ISBN: 0818689307). The k-NN computes the distance of a test sample to each of the training set samples, each of which has an associated class (in this case, Survivor or Failure), and then predicts the class of the test sample to be that of the majority of the k closest samples. The k-NN classifier was evaluated by cross-validation, whereby one sample is randomly withheld, a model is trained on the remaining samples, and the model is then used to predict the class of the withheld sample. The process is repeated until all of the samples are tested.

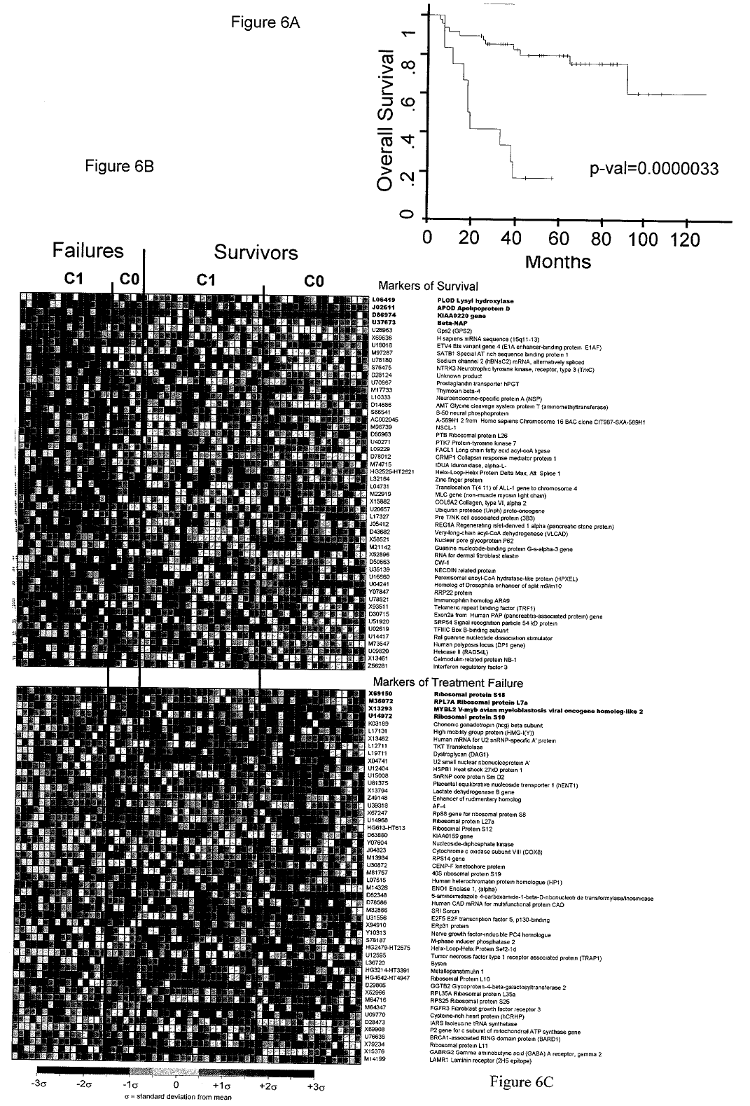

Gene expression-based outcome predictions were statistically significant for k-NN models ranging from 2 to 21 genes, with optimal predictions made by an 8-gene model which made only 13/60 classification errors (Fisher Exact Test P=0.0002). Shown most clearly by Kaplan-Meier survival analysis in FIG. 6A, patients predicted to be Survivors had a 5-year overall survival of 80% compared to 17% for patients predicted to have a poor outcome (P=0.000003, log-rank test). A more conservative method of assessing statistical significance is to attempt to optimize classifiers of random permutations of the Survivor/Failure class labels. 1000 such permutations were determined, and only 9/1000 permutations were found for which prediction accuracy matched or exceeded our observed result (Supplementary Information Section III; http://www.genome.wi.mit.edu/MPR/CNS), indicating that the result is unlikely to be achieved by chance (P=0.009). Therefore, several other classification algorithms including Weighted Voting were subsequently tested (Golub, T. et al., 1999. Science, 286:531-537; Slonim, D. et al., 2000. Procs. of the Fourth Annual International Conference on Computational Molecular Biology, Tokyo, Japan Apr. 8-11, p263-272, 2000), Support Vector Machines (Mukherjee, S. et al., 1999. Support vector machine classification of microarray data. CBCL Paper #182/AI Memo #1676, Massachusetts Institute of Technology, Cambridge, Mass.; Brown, M. et al., 2000. Proc. Natl. Acad. Sci. USA, 97:262-267), and IBM SPLASH (Califano et al., Proceedings of the Eighth International Conference on Intelligent Systems for Molecular Biology, San Diego, Calif., Aug. 19-23, p75-85, 1999), all of which performed with similarly high accuracy (Supplementary Information, Sections I and III; http ://www.genome.wi.mit.edu/MPR/CNS).

The clinical value of the predictor was explored further by considering existing prognostic factors for medulloblastoma outcome. Patients with localized disease (MO) had a more favorable outcome compared to patients with involvement of the cerebrospinal fluid or with distant metastases (M+) (P=0.03 comparing M0 with M+ by Kaplan-Meier analysis), although not all M0 patients survived. When the outcome predictor was applied only to the 42 M0 patients, the prediction of outcome remained significant (P=0.002), indicating that the expression-based predictor substantially improved staging-based prognostication. Similarly, TrkC-based prediction was imperfect in this series in that not all patients in the unfavorable (TrkC-low) category died. When the gene expression-based predictor was applied to the 33 TrkC-low patients, the surviving patients could be significantly separated from those who succumbed to their disease (P=0.01; Supplementary Information Section III; http://www.genome.wi.mit.edu/MPR/CNS). Of note, not all patients in this study received identical therapy. However, restricting the analysis to the 35 patients that received surgery, vincristine, cisplatin and cyclophosphamide, the predictor continued to yield a significant Kaplan-Meier survival distinction (P=0.0012). Taken together, these results demonstrate that the gene expression-based outcome predictor exceeds other approaches to prognosis determination.

A number of genes not previously associated with clinical outcome were identified (FIG. 6B and 6C). Those correlated with favorable outcome included many genes characteristic of cerebellar differentiation (vesicle coat protein beta-NAP, NSCL-1, TrkC, sodium channels), and genes encoding extracellular matrix proteins (PLOD lysyl hydroxylase, collagen type VIα, elastin). As expected, TrkC expression was correlated with a favorable outcome, consistent with prior reports of this association (Segal, R. et al., 1994. Proc. Natl. Acad. Sci. USA, 91:12867-12871; Kim, J. et al., 1999. Cancer Res., 59:711-719; Grotzer, M. et al., 2000. J Clin. Oncol., 18:1027-1035). In contrast, genes related to cerebellar differentiation were under-expressed in poor prognosis tumors, which were dominated by the expression of genes related to cell proliferation and metabolism (MYBL2, enolase 1, LDH, HMG-I(Y), cytochrome C oxidase) and multidrug resistance (sorcin). Genes correlated with poor outcome included a number of the ribosomal protein-encoding genes identified by the SOM clustering experiments (FIG. 6B and 6C). This indicates that whereas this ribosomal signature is correlated with poor outcome, optimal outcome prediction requires not only these genes, but also genes correlated with a favorable outcome, which were not identified by the unsupervised clustering analysis.

For patients predicted to have a favorable outcome, efforts to minimize toxicity of therapy might be indicated, whereas for those predicted not to respond to standard therapy, earlier treatment with experimental regimens might be considered.

Methods

Patient Samples. Patients included 60 children with medulloblastoma, 10 young adults with malignant glioma (WHO grades III and IV), 5 children with AT/RT, 5 with renal/extrarenal rhabdoid tumors, and 8 children with supratentorial PNET (see Supplementary Information Section I; http://www.genome.wi.mit.edu/MPR/CNS). Medulloblastoma patients were treated with craniospinal irradiation to 2400-3600 centiGray (cGy) with a tumor dose of 5300-7200 cGy. All patients with medulloblastoma were treated with chemotherapy consisting of cisplatin and vincristine, plus combinations of carboplatin, etoposide, cyclophosphamide or lumustine (CCNU) (details in Supplementary Information Section II; http://www.genome.wi.mit.edu/MPR/CNS). Samples were snap frozen in liquid nitrogen and stored at −80° C. Studies were done with approval of the Committee for Clinical Investigation of Boston Children's Hospital. The data were organized into three sets: Dataset A (42 samples containing 10 medulloblastoma, 10 malignant glioma, 10 AT/RT, 8 PNET and 4 normal cerebellum), Dataset B (34 samples, containing 9 desmoplastic medulloblastoma and 25 classic medulloblastoma), and Dataset C (60 samples, containing 39 medulloblastoma survivors and 21 treatment failures). The clinical attributes of each of the patients in the study are available in Supplementary Information Section II (http://www.genome.wi.mit.edu/MPR/CNS). Tissues were homogenized in guanidinium isothiocyanate and RNA was isolated by centrifugation over a CsCl gradient. RNA integrity was assessed either by northern blotting or by gel electrophoresis. 10-12 μg total RNA was used to generate biotinlylated antisense RNAs which were hybridized overnight to HuGeneFL arrays containing 5920 known genes and 897 expressed sequence tags as previously described (Golub, T. et al., 1999. Science, 286:531-537). Arrays were scanned on Affymetrix scanners and the expression value for each gene was calculated using Affymetrix GENECHIP software. Minor differences in microarray intensity were corrected using a linear scaling method as detailed in Supplementary Information Section I (http://www.genome.wi.mit.edu/MPR/CNS). Scans were rejected if the scaling factor exceeded 3, fewer than 1000 genes received ‘Present’ calls, or microarray artifacts were visible.

Data Analysis: Preprocessing. The gene expression data were subjected to a variation filter that excluded genes showing minimal variation across the samples being analyzed, as detailed in Supplementary Information Section I (http://www.genome.wi.mit.edu/MPR/CNS).

Data Analysis: Clustering. The data were first normalized by standardizing each column (sample) to mean 0 and variance 1. SOMs were performed using the GeneCluster clustering package available at www.genome.wi.mit.edu/MPR/Software. Hierarchical clustering was performed using Cluster and TreeView software (Eisen, M. et al., 1998. Proc. Natl. Acad. Sci. USA, 95:14863-14868). PCA was performed by computing and then plotting the 3 principal components using the S-Plus statistical software package using default settings.

Data Analysis: Supervised Learning. Genes correlated with particular class distinctions (e.g., classic vs. desmoplastic medulloblastoma) were identified by sorting all of the genes on the array according the signal-to-noise statistic (μ₀−μ₁)/(σ₀+σ₁), where μ and σ represent the median and standard deviation of expression, respectively, for each class. Similar results were obtained using a standard t-statistic as the metric ((μ_{0-μ}₁)/sqrt(σ₀²/N0+σ₁²/N₁)), where N represents the number of samples in each class (see Supplementary Information; http://www.genome.wi.mit.edu/MPR/CNS). Permutation of the column (sample) labels was performed to compare these correlations to what would be expected by chance in 99% of the permutations. For classification, a modification of the k-NN algorithm was developed that predicts the class of a new data point by calculating the Euclidean distance (d) of the new sample to the k nearest samples (for these experiments, k=5) in the training set using normalized gene expression data, and selecting the class to be that of the majority of the k samples. The weight given to each neighbor was 1/d. The k-NN models were evaluated by 60-fold leave-one-out cross-validation whereby a training set of 59 samples was used to predict the class of a randomly withheld sample, and the cumulative error rate was recorded. Models with variable numbers of genes (1-200, selected according to their correlation with the survivor vs. treatment failure distinction in the training set) were tested in this manner. An 8-gene k-NN outcome prediction model yielded the lowest error rate, and was therefore used to generate Kaplan-Meier survival plots using S-Plus. Predictors using metastatic staging or TrkC were constructed by finding the decision boundary half way between the classes: (μ_{class0}+μ_{class1})/2 using either the staging values 0 vs. 1, 2, 3, 4 or the continuous TrkC microarray gene expression levels, and then predicting the unknown sample according to its location with respect to that boundary.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of classifying a brain tumor comprising the steps of:
a) obtaining a sample of cells derived from a brain tumor;
b) isolating a gene expression product from at least one informative gene from one or more cells in said sample; and
c) determining a gene expression profile of at least one informative gene, wherein the gene expression profile is correlated with a specific brain tumor subtype.

2. The method of claim 1, wherein the brain tumor type is selected from the group consisting of: medulloblastoma, rhabdoid tumor, primitive neuroectodermal tumor, pineoblastoma and glioblastoma.

3. The method of claim 2, wherein the brain tumor type is a medulloblastoma or a glioblastoma.

4. The method of claim 3, wherein the medulloblastoma sub-type is classic medulloblastoma or desmoplastic medulloblastoma.

5. The method of claim 2, wherein the expression profile comprises expression of Zic or NSCL-1.

6. The method of claim 1, wherein the expression profile comprises expression of TrkC.

7. The method of claim 1, wherein the gene expression product is mRNA.

8. The method of claim 7, wherein the gene expression profile is determined utilizing specific hybridization probes.

9. The method of claim 7, wherein the gene expression profile is determined utilizing oligonucleotide microarrays.

10. The method of claim 1, wherein the gene expression product is a polypeptide.

11. The method of claim 10, wherein the gene expression profile is determined utilizing antibodies.

12. A method according to claim 1, wherein one or more informative genes is selected from the group consisting of the genes in FIGS. 2A-2B, 3A-3B, 5A-5B and 6B-6C.

13. A method according to claim 1, wherein one or more informative genes is selected from the group consisting of the genes in FIGS. 1A-1B.

14. A method of predicting the efficacy of treating a brain tumor comprising the steps of:
a) obtaining a sample of cells derived from a brain tumor;
b) isolating a gene expression product from at least one informative gene from one or more cells in said sample; and
c) determining a gene expression profile of at least one informative gene,
wherein the gene expression profile is correlated with a treatment outcome, thereby classifying the sample with respect to treatment outcome.

15. The method of claim 14, wherein the brain tumor type is selected from the group consisting of: medulloblastoma, rhabdoid tumor, primitive neuroectodermal tumors, pineoblastoma and glioblastoma.

16. The method of claim 15, wherein the brain tumor type is a medulloblastoma or a glioblastoma.

17. The method of claim 16, wherein the medulloblastoma sub-type is classic medulloblastoma or desmoplastic medulloblastoma.

18. A method according to claim 14, wherein the gene expression product is mRNA.

19. A method according to claim 18, wherein the gene expression profile is determined utilizing specific hybridization probes.

20. A method according to claim 18, wherein the gene expression profile is determined utilizing oligonucleotide microarrays.

21. A method according to claim 14, wherein the gene expression product is a polypeptide.

22. A method according to claim 21, wherein the gene expression profile is determined utilizing antibodies.

23. A method according to claim 14, wherein the predicted treatment outcome is survival after treatment.

24. A method according to claim 14, wherein one or more informative genes is selected from the group consisting of the genes in FIGS. 1A-1B.

25. A method according to claim 14, wherein one or more informative genes is selected from the group consisting of the genes in FIGS. 2A-2B, 3A-3B, 5A-5B and 6B-6C.

26. A method of assigning a brain tumor sample to a treatment outcome class, comprising the steps of:
a) determining a weighted vote for one of the classes of one or more informative genes in said sample in accordance with a model built with a weighted voting scheme, wherein the magnitude of each vote depends on the expression level of the gene in said sample and on the degree of correlation of the gene's expression with class distinction; and
b) summing the votes to determine the winning class,
wherein the winning class is the treatment outcome class to which the brain tumor sample is assigned.

27. The method of claim 26, wherein the weighted voting scheme is:
V_{g}=a_{g}(x_{g}−b_{g}),
wherein V_{g} is the weighted vote of the gene, g; a_{g} is the correlation between gene expression values and class distinction; b_{g}=(μ₁(g)+μ₂(g))/2 is the average of the mean log₁₀ expression value in a first class and a second class; x_{g} is the log₁₀ gene expression value in the sample to be tested; and wherein a positive V value indicates a vote for the first class, and a negative V value indicates a vote for the second class.

28. The method according to claim 26, wherein the informative genes are selected from the group consisting of the genes in FIGS. 1A-1B.

29. The method according to claim 26, wherein the informative genes are selected from the group consisting of the genes in FIGS. 2A-2B, 3A-3B, 5A-5B and 6B-6C.

30. An oligonucleotide microarray having immobilized thereon a plurality of oligonucleotide probes specific for one or more informative genes selected from the group consisting of the genes in FIGS. 1A-1B, 2A-2B, 3A-3B, 5A-5B and 6B-6C.

31. A method for evaluating drug candidates for their effectiveness in treating brain tumors comprising:
a) obtaining samples of cells derived from a brain tumor;
b) isolating a gene expression product from at least one informative gene from one or more cells in said samples; and
c) determining a gene expression profile of at least one informative gene,
wherein the gene expression profile is correlated with the effectiveness of the drug candidate in treating brain tumors.

32. A method for monitoring the efficacy of a brain tumor treatment comprising:
a) obtaining samples of cells at various time points derived from a patient being treated;
b) determining the expression profile of the samples;
c) classifying the samples for treatment outcome based on the expression profile; and
d) comparing the treatment outcome class of the samples at various times during treatment,
wherein the efficacy of brain tumor treatment is determined.

33. A method for predicting tumorigenesis comprising:
a) obtaining samples of cells at various time points derived from a patient;
b) determining the expression profile of the samples;
c) classifying the samples as tumorigenic or non-tumorigenic based on the expression profile; and
d) comparing the tumorigenic class of the samples at various times, such that the onset of tumorigenesis can be predicted.
